(19) United States
(12) Patent Application Publication
(10) Pub. No.: US 2007/0011781 A1
(43) Pub. Date: Jan. 11, 2007

(54) Male sterility in grasses of the genus Lolium

(76) Inventors: Inge Gaue, Kaltenhof (DE); Heinrich Baudis, Malchow (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/520,186

(22) Filed: Sep. 12, 2006

Related U.S. Application Data
(62) Division of application No. 10/764,056, filed on Jan. 23, 2004, now abandoned.
Application No. 10/764,056 is a continuation of application No. PCT/EP02/08252, filed on Jul. 24, 2002.

(30) Foreign Application Priority Data
Jul. 26, 2001 (DE) 101 36 378.8

(51) Int. Cl. A01H 5/00; C12Q 1/68; C12N 15/82; A01N 47/28; C12N 5/04; A01H 1/00

(52) U.S. Cl. 800/287; 800/320; 435/419; 435/468; 435/6; 504/327

(57) ABSTRACT

A method of producing male sterile plants of the genus Lolium is provided. The method involves the mutagenesis of wild-type plants, followed by the identification of completely male sterile plants, preferably by methods such as pollen vitality measurements or by molecular biology techniques. The invention also includes the male sterile plants produced by this method. The male sterile plants so produced are completely sterile and are useful for the production of hybrid seeds and plants. Also disclosed are the hybrid seeds and plants produced using the male sterile plants of the invention.

### RELATED APPLICATIONS

This application is a divisional of U.S. patent application Ser. No. 10/764056, filed Jan. 23, 2004, which is a continuation of International Application No. PCT/EP02/08252, filed Jul. 24, 2002, which claims priority to German Patent Application No. 101 36 378.8, filed Jul. 26, 2001, the disclosures of which are incorporated by reference herein in their entireties.

### BACKGROUND OF THE INVENTION

1. Field of the Invention

The invention relates to a method for producing stable male sterile plants of the genus Lolium for use in the specific production of hybrid varieties by utilization of heterosis.

2. Description of the Related Art

Plants, as eukaryotes, have two or more copies of their genetic information per cell. Each gene is usually represented by two alleles, which can be identical in the homozygous condition or different in the heterozygous condition. When two selected inbred lines are crossed, the F1 hybrids produced in the first generation, i.e. heterozygous individuals, are often bigger, more robust and also more productive than the homozygous parents, presumably because both of their allelic gene products a) are less likely to be inactivated, or b) they have a greater reactivity. This effect, called heterosis or hybrid vitality, has already been exploited by plant breeders for many decades for the production of hybrid varieties.

Thus, the phenomenon of heterosis is understood to mean the increase of quantitative feature presentations in progeny beyond the average of the parents or the performance of the superior parent: In particular, the growth (plant length, degree of branching, etc.) and the yield as well as the quantitatively inherited characteristics (for instance resistance) may be affected. The breeding of hybrid lines is carried out using cytoplasmic male sterility (CMS) or self-incompatibility (SI), the two most important genetic systems for inhibiting self-pollination. A total utilization of heterosis can be achieved by exploiting the cytoplasmic inheritable male sterility (C. Bothe, Nutzung von teilfertilen ms-Linien für die Züchtung von Chance-Hybriden bei Welschem Weidelgras (Lolium multiflorum Lam.), Dissertation Göttingen, Cuvillier Verlag Göttingen 1996, 113 S., G. Kobabe, Heterosis and hybrid seed production in fodder grass, Monographs on Theoretical and Applied Genetics, Vol. 6 Heterosis, Editor: R. Frankel, Springer Verlag Berlin Heidelberg 1983; V. Lein, Heterosis in Kreuzungen zwischen Inzuchtlinien des Deutschen und Welschen Weidelgrases (Lolium perenne L.×Lolium multiflorum Lam. ssp. italicum) Dissertation Göttingen 1988; 91 S.) By one breeding partner losing its ability to produce fertile pollen, cytoplasmic male sterility enables the selective production of F₁ hybrids. Female fertility is not affected.

In the breeding of cultivated plants with improved agronomical performance and adapted content, different breeding methods may be used depending on the natural mode of pollination of the respective plant species. While for the strict self-pollinators such as barley (Hordeum vulgare L.) and wheat (Triticum aestivum L.) methods of line breeding are used, for obligatory cross-pollinators such as rye (Secale cereale L.) population breeding, synthetic breeding and hybrid breeding has been used in the past, for which heterosis being increasingly used.

The plant material available for breeding possesses a natural variability in the degree of heterosis. A distinction is made here between a general combining ability (GCA) and specific combining ability (SCA). Lines with a high GCA are characterized by a high heterotic performance in breedings with different parent lines. Plant lines with a high SCA show a high heterotic performance in combination with a specific breeding partner. The SCA can therefore only be determined in comparative pairwise breedings (e.g. diallelic).

With respect to the utilization of heterosis, usually plant material is selected for breeding which is characterized by a good natural contribution and a good GCA or SCA.

While for the breeding of population varieties first mass selection, i.e. repeated selection of the best individual plants and joint continuation to a homogenous variety is initially pursued, for the production of so-called synthetic varieties (synthetics) several lines are selected, which are characterized by a good GCA. For seed production, different lines are then selectively cultivated together for two to three generations until they are marketed as seeds. These synthetics usually show a higher degree of heterosis and thus especially a higher yield than the population varieties.

Hybrid varieties are a further progression in the utilization of heterosis in plant varieties. By the selective combination of specific parent lines with good GCA or SCA in the last step of seed production hybrid varieties can be produced which are characterized by a very high heterosis performance and thus a noticeably increased yield.

A precondition for the production of hybrid varieties is the directed pollination of a mother line with a selected father line as pollen donor. In order to produce sufficient amounts of seeds, the latter step has to be performed on large-scale under outdoor conditions. By cultivating a pollenless, i.e. male sterile, mother line and a fertile (pollen-bearing) father line in direct proximity, such a directed pollination is achieved.

Thereby hybrid seeds are created to a large extent, which can be solely traced back to the crossing combination of the two parent lines. A precondition for this is particularly the existence of a complete, i.e. if possible 100% male sterile mother line which cannot pollinate itself.

For this reason various methods were developed in the past, in particular mechanical, chemical and genetic methods for the induction of male sterility of plants. Mechanical methods, such as for instance the removal of the anthers, are only suitable for plant species having large and/or spatially separated sexual organs, such as for instance corn (Zea mays L.). For the chemical emasculation of plants substances called gametocides were developed, which have a lethal effect on pollen after application. In this way, also hybrid varieties of strict self-pollinators such as wheat and barley could be produced for the first time.

Genetic mechanisms which induce male sterility of plants have been previously described. For instance, male sterile plants basically occur in the mostly aneuploid progeny of wide, i.e. inter-specific or intergeneric crossings. This is partly due to irregularities in the meiosis of the progeny and affects both male and female gametes to the same extent. In addition, also systems have been discovered and further refined, which are based on single gene defects and which only influence the male gametes and the pollen. Such systems can be traced back on the one hand to mutations in the nuclear genome (nuclear male sterility, NMS) and on the other hand to gene alterations in the plastom or cytoplasm (cytoplasmic male sterility, CMS).

The CMS is based in principle on the incompatibility of the nucleus and the cytoplasm and is inherited strictly maternally in most higher plants (U. Witt, Identifikation und Charakterisierung eines kernkodierten Mitochondrienproteins aus dem pollensterilität-induzierenden Polima-Cytoplasma von Brassica napus L., Dissertation, Hamburg 1993).

With the help of a corresponding father line, which is not able to overcome the sterility of the maternal cytoplasm (so-called “maintainer”), theoretically a homozygous sterile CMS plant may be produced after repeated back-crossing with the maintainer plant. A complete CMS system, for example for the production of hybrid seeds of grasses whose vegetative mass is used, thus consists of the following components:
1. the CMS line which bears a sterility-inducing cytoplasm (S), also called sterile mother line.
2. the maintainer line, which bears a normal fertile cytoplasm (N) and which is very similar to the CMS line in other respects.
3. the pollinator line or father line, which is normally fertile and which is suitable for combination with the CMS mother line.

A fundamental technical problem for the production of hybrid varieties is the stability of the male sterility of the CMS line. This particularly affects the 100% transfer of the male sterility to the next generation after crossing and the provision of an environmentally independent phenotype in the form of male sterile plants. Only under these conditions can agronomically optimized and complete male sterile mother plants be generated, which permit heterosis in the form of hybrid varieties to be exploited to its full extent and to realize an additional yield potential.

The Lolium species perennial ryegrass (Lolium perenne L.), annual ryegrass (Lolium multiflorum L.) and hybrid ryegrass (Lolium hybridum L.) are the most important grass species in European food grass culture. For food grasses the specific exploitation of heterosis effects is thought to be a real possibility for substantially increasing yields and for improving further quantitative characteristics such as stress tolerances against biotic and abiotic factors. As the aforementioned Lolium species are cross-pollinators, the breeding of synthetics and hybrid varieties presents itself for this purpose.

In order to achieve additional variability as a basis for the selection of new genotypes, the method of polyploidization is used in the breeding of cultured plants. With polyploidization, by using mitosis inhibitors such as colchicine during mitosis the chromosome set of a cell is doubled. In the case of Lolium species this leads to the generation of tetraploid forms from originally diploid species (2n=2x=14), which have a double chromosome set (2n=4x=28). Because tetraploids possess other characteristics besides diploids, for the economically relevant Lolium species L. perenne, L. multiflorum and L. hybridum corresponding tetraploid varieties have been cultivated.

For ryegrass species there are a number of studies which point to heterosis and hybrid growth in all valences (including C. A. Foster, Interpopulational and intervarietal hybridization in Lolium perenne breeding, heterosis under noncompetitive conditions, J. Agric. Sci. 1971, 107-130; C. A. Foster, (1973): Interpopulational and intervarietal F₁-Hybrids in Lolium perenne: performance in field sward conditions, J. Agric. Sci. 1973, 80, 463-477; I. Rod, Beitrag zu den methodischen Fragen der Heterosiszüchtung bei Futtergräsern, Ber. Arbeitstagung Arbeitsgemeinsch. Saatzuchtleiter Gumpenstein 1965, pages 235-252; I. Rod, Remarks on heterosis with grasses, Heterosis in plant breeding, Proc. 7th Congr. Eucarpia Budapest (1967), pages 227-235; A. J. Wright, A theoretical appraisal of relative merits of 50% hybrid and synthetic, J. Agric. Sci. 79, 1972, pages 245-247). In the past, heterosis effects could be detected especially after single plant crossings, line crossings and variety crossings (Kobabe, see above).

The breeding method most commonly used at present, namely the production of synthetics or varieties on the basis of clones or populations, was developed for grasses by Frandsen (N. H. Frandsen, Some breeding experiments with timothy, Imp. Agric. Bur. Joint Publ. 1940, 3, 80-92) in 1940. However, as mentioned above, this method only allows a partial use of heterosis. A true food grass hybrid variety by using a Lolium line with cytoplasmic male sterility for the complete utilization of heterosis (C. Bothe, see above; G. Kobabe, see above; V. Lein, see above) is not known so far, because no plants with complete male sterility were available and the known CMS sources are unstable.

The systems found or used for Lolium species for the achievement of male sterility differ with respect to their origin and mode of action. Systems with mechanical control for the castration of the plants are ruled out for Lolium species due to their morphology. Chemical methods have not yet been developed for Lolium, while genetic control mechanisms were described previously. Spontaneously generated sources have been reported by Nitzsche (Cytoplasmatische männliche Sterilität bei Weidelgras (Lolium ssp.) Z. Pflanzenzücht., Berlin (West) 65, (1971), pages 206-220) for Lolium multiflorum, and, for Lolium perenne, by Gaue (Möglichkeiten der Hybridzüchtung auf ms-Basis bei Lolium perenne L. XIII. Internat. Grasland-Kongreβ, Leipzig 1977, Sektionsvortrag 1-2, pages 491-496; Ergebnisse von Untersuchungen zur Hybridzüchtung bei Lolium perenne Tag.-Ber., Akad. Landwirtsch.-Wiss. DDR, Berlin (1981) 191, pages 119-126). After species and genus crossings male sterile forms also developed for Lolium perenne (F. Wit, Cytoplasmic male sterility in ryegrasses (Lolium ssp.) detected after intergeneric hybridization, Euphytica 1974, 23, 31-38; V. Connoly, Hybrid grasses varieties for the future Farm Food Res. 1978, 9, 6, 131-132; V. Connoly, R. Wright-Turner, Induction of cytoplasmic male-sterility into ryegrass (Lolium perenne), Theor. Appl. Genet. 1984, 68, 449-453). However, none of these genetic systems could be stabilized genotypically and phenotypically, so that up to now no functional hybrid system is known for the different ryegrass species.

Although the production of hybrid lines with improved agronomic characteristics is intensively studied, methods available so far for the production of male sterile plants do not lead to completely satisfactory results in many cases. There is therefore a strong need for a method for the production of completely male sterile and stable plants which do not show the disadvantages of the prior art.

### SUMMARY OF THE INVENTION

In some embodiments, a method for the production of completely male sterile plants of the genus Lolium is provided, by mutagenizing caryopses material of wild-type plants of the genus Lolium; and identifying at least one completely male sterile Lolium plant. Optionally, the mutagenized Lolium plants can be examined by at least one test method. The test method can be, for example, directed to pollen vitality or a molecular biological method. The mutagenesis procedure can be performed by addition of chemical mutagens, such as, for example, N-ethyl urea. The Lolium plants can be, for example, Lolium perenne, Lolium multiflorum, or Lolium hybridum.

Pollen vitality can be measured, for example, using staining methods. Examples of such staining methods include, for example, the method according to Alexander, the addition of light green reagent, the addition of Lugol's solution, and the like. In additional aspects, the mutagenized Lolium plants can be examined by Southern Blot techniques. In some aspects of the invention, the method employs primer pairs for amplification of probes used for Southern Blot hybridization, using, for example, at least one of the following primer pairs:

| | | | |
|---|---|---|---|
| a) | upper: | TTACTTCACATAGCTTTTCGTU | (SEQ ID NO. 1) |
| | lower: | CCACAAACCACAAGGATATAG | (SEQ ID NO. 2) |
| b) | upper: | ATGATTGAATCTCAGAGGCAT | (SEQ ID NO. 5) |
| | lower: | CATATACCTCCCCACCAATAG | (SEQ ID NO. 6) |
| c) | upper: | TTAGTAGATCGTGAGTGGGTC | (SEQ ID NO. 7) |
| | lower: | GTGCTAAAAATCCGGTACAT | (SEQ ID NO. 8) |
| d) | upper: | TTATCCGTCGCTACGCTGTTC | (SEQ ID NO. 9) |
| | lower: | AATGGAAAGATCGGAACATGG | (SEQ ID NO. 10) |
| e) | upper: | ATGACTATAAGGAACCAACGA | (SEQ ID NO. 17) |
| | lower: | GATCAGTCTCATCCGTGTAA | (SEQ ID NO. 18) |
| f) | upper: | ATGAGACGACTTTTTCTTGAA | (SEQ ID NO. 19) |
| | lower: | CTTGTAAACTAATCGAGACCG. | (SEQ ID NO. 20) |

The probes that are prepared can be used for southern blot analysis, preferably by digesting the sample DNA with one of the following combinations of restriction enzymes: a) HindIII or DraI; b) HindIII, DraI or EcoRV; c) HindIII or BamHI; d) HindIII, XbaI, DraI, EcoRV, BamHI or HaeII; e) XbaI or HaeIII; or f) EcoRV.

In additional embodiments of the present invention, any of the above methods can be used for the production of stable F₁ hybrids of completely male sterile plants of the genus Lolium, by producing a completely male sterile plant of the genus Lolium (MSL plants), and back-crossing the MSL plant thus obtained with one or more plants of the genus Lolium, which have normal fertile cytoplasm and which maintain the sterility of the MSL plants (maintainer plants). In some aspects, the MSL plants are identified by at least one test method directed to pollen vitality or a molecular biological method. In additional aspects, plants of the corresponding species are used as maintainer plants, which lead to a 100% pollen-sterile progeny after crossing with the MSL line. In some aspects, a multiple back-crossing with maintainer plants is performed.

Furthermore, the sterility-inducing plasm of the MSL plant produced by any of the above methods can be brought to a preferably tetraploid valence by polyploidization. This can be achieved, for example, using a colchicine treatment. In further aspects, the method can be used to produce Lolium plants with complete male sterility. Accordingly, in some embodiments of the invention, Lolium plants with complete male sterility are provided.

Also, some embodiments relate to, for example, methods for the production of hybrids with pollinator plants having normal male fertility, using the completely male sterile plants of the genus Lolium according to any of the above methods. Additional embodiments relate to, for example, hybrid seeds produced by any of the above methods.

### BRIEF DESCRIPTION OF THE DRAWINGS

FIG. 1: A, pollen of an individual plant CMS-1 (S) after KES staining; B, pollen of an individual plant CMS-1 (N) after KES staining (magnification scales are not identical).

FIG. 2: A, restriction digest of total DNA (a) and mtDNA (b) of the plants MSL-19, MSL-163 and CMS-1 with the restriction enzyme HindIII; B, Southern hybridization with the probe/enzyme combination nad9/HindIII; C, Southern hybridization with the probe/enzyme combination Actin/HindIII; (S)=CMS plant; (N)=maintainer.

FIG. 3: Southern hybridization (probe/enzyme combination nad9/DraI) of DNA bulks of the plant Inca (CMS-I1, CMS-I2, CMS-I3, CMS-I4) and CMS-1 (S), MSL-163 (S) and MSL-19 (S) and of individual plants of the sources CMS-113, CMS-114, CMS-115, CMS-117, CMS-118 and CMS-119.

### DETAILED DESCRIPTION OF THE PREFERRED EMBODIMENT

It is therefore an object of the present invention to provide methods for the production of stable, i.e. among other things, environmentally independent and completely, i.e. 100%, male sterile plants of the genus Lolium, which enable the specific production of hybrid plants and thus utilization of the effects of heterosis such as for example additional yield.

This and other objects of the invention are solved by the provision of embodiments characterized in the patent claims.

According to the invention a method is provided by which it is possible for the first time to produce completely, i.e. 100% male sterile plants of the genus Lolium (hereinafter also referred to as MSL or MSL plant (male sterility Lolium). This completely new plant of the genus Lolium is characterized by a high stability of the sterility-inducing plasm. In particular, the male sterility in the plant produced by the method according to the invention is temperature-stable and shows only an extremely low environmental dependency. In contrast to the sterile crossing progeny of plants with male sterility known up to now, in which, depending on the anther shape, a high degree of partially sterile plants is present, the degree of sterility in MSL is uniformly high, irrespective of the anther shaping (Tables 2 to 4). The MSL lines produced by the method according to the invention consequently for the first time enable the production of homogenous Lolium-F₁ hybrid varieties under outdoor conditions.

The method according to the invention for the production of completely male sterile plants of the genus Lolium comprises the following steps:
a) mutagenesis of seed material of wild-type plants of the genus Lolium;
b) optionally, examination of the mutagenized Lolium plants with test methods directed to the pollen vitality and/or by molecular biological methods; and
c) identification of completely male sterile Lolium plants.

Within the scope of the invention, wild-type plants are understood to mean naturally occurring plants of the genus Lolium, especially those whose genetic information has not been manipulated by mutagenesis. The seed material is especially caryopses material.

The mutagenesis in step a) of the method according to the invention is preferably performed by treatment of the seed material with chemical mutagens. N-ethyl urea is especially preferred for this purpose. Other possible mutagens are alkylating agents such as ethane sulfonate and diepoxy butane, urethane, nitroso compounds, alkaloids such as colchicine, peroxides such as H₂O₂, alkyl peroxides and the like.

In an alternative embodiment mutagenesis is carried out by irradiation of seed material with shortwave UV light (e.g. 254 nm); longwave UV light (e.g. 300 to 400 nm) in combination with psoralens; ionising radiation such as X-ray and γ irradiation; and the like.

The Lolium plants, in which the cytoplasmic male sterility is produced, are preferably selected from the group consisting of Lolium perenne, Lolium multiflorum and Lolium hybridum.

The analysis of mutagenized Lolium plants using test methods directed to the pollen vitality serves the purpose of differentiating between fertile pollen and the desired sterile pollen. Because of the good correspondence between anther shape and degree of sterility—99% of the plants produced by the method according to the invention which were visually classified as sterile, are indeed completely sterile—analyses of pollen vitality are only necessary in the method according to the invention if a control is desired. For the plants with male sterility known from the prior art, no reliable visual evaluation of the male sterility produced is possible, because anther shape and degree of sterility do not correspond, or correspond only to a lesser extent (Table 3).

Preferably, the test methods are staining methods, such as for example the method according to Alexander (M. P. Alexander, Differential staining of aborted and non-aborted pollen, Stain Technology 1969, 44/3, 117-122), the addition of light green reagent (I. \check{S}inska, Ergebnisse der Forschung der Pollensterilität der Luzerne d'Eucarpia-Groupe Medicago sativa Piestany 17.-21.5.1976) and the addition of Lugol's solution. The corresponding reagents for the above-mentioned staining methods are shown in Table 1.

Alternatively, or in addition to the above-described analyses of the mutagenized Lolium plants with respect to pollen vitality, for example by visual investigation or staining methods, molecular biological methods for the reliable genotypic differentiation between MSL and Lolium plants which do not show complete male sterility may be used. Especially preferred molecular biological differentiation is performed by Southern Blot techniques.

Primer pairs for the amplification of the probes used for the Southern Blot hybridization are preferably selected from the group consisting of the following primer pairs (see also Table 6):

| | | |
|---|---|---|
| a) | TTACTTCACATAGCTTTTCGTU | (SEQ ID NO. 1) |
| and | | |
| | CCACAAACCACAAGGATATAG; | (SEQ ID NO. 2) |
| b) | CGTAAAGGCATGATTAGTTCC | (SEQ ID NO. 3) |
| and | | |
| | GATTGTTCTAAAATGGTTATTCCTC; | (SEQ ID NO. 4) |
| c) | ATGATTGAATCTCAGAGGCAT | (SEQ ID NO. 5) |
| and | | |
| | CATATACCTCCCCACCAATAG; | (SEQ ID NO. 6) |
| d) | TTAGTAGATCGTGAGTGGGTC | (SEQ ID NO. 7) |
| and | | |
| | GTGCTAAAAATCCGGTACAT; | (SEQ ID NO. 8) |
| e) | TTATCCGTCGCTACGCTGTTC | (SEQ ID NO. 9) |
| and | | |
| | AATGGAAAGATCGGAACATGG; | (SEQ ID NO. 10) |
| f) | ATGTTTCCACTCAATTTTCAT | (SEQ ID NO. 11) |
| and | | |
| | GCTCCACAGTGGTAAAGTCT; | (SEQ ID NO. 12) |
| g) | TTACGACCACTGAACAAACTT | (SEQ ID NO. 13) |
| and | | |
| | TTTAACCATAAAATCGATTATGC; | (SEQ ID NO. 14) |
| h) | CTATATTTCGTACGTTTCGGA | (SEQ ID NO. 15) |
| and | | |
| | TTATTATGGTAAATTTGTGTATCAA; | (SEQ ID NO. 16) |
| i) | ATGACTATAAGGAACCAACGA | (SEQ ID NO. 17) |
| and | | |
| | GATCAGTCTCATCCGTGTAA; | (SEQ ID NO. 18) |
| j) | ATGAGACGACTTTTTCTTGAA | (SEQ ID NO. 19) |
| and | | |
| | CTTGTAAACTAATCGAGACCG; | (SEQ ID NO. 20) |

k) primer pairs according to the primer pairs shown in a) to j), wherein the corresponding primer sequences differ from the sequences shown in a) to j) by a maximum of 3 bases each, as well as
l) primer pairs corresponding to the primer pairs shown in a) to k), wherein the primer sequences comprise the sequences shown in a) to k),
wherein the sequences are shown in 5′-3′ direction and the first sequence is the upper primer.

Restriction enzymes preferred for the Southern Blot hybridization in the method according to the invention are selected from the group consisting of HindIII, XbaI, DraI, EcoRV, BamHI and HaeIII.

By combining the above-described probes and enzymes, particularly by combination of the probe nad9 (see Table 6) with the restriction enzyme DraI, it is possible to clearly distinguish between plants with stable and instable male sterility and complete and incomplete male sterility, respectively.

In another essential aspect of the present invention, plants of the genus Lolium with complete male sterility are provided, which can be produced according to the method described above.

A further subject of the invention is a method for the production of stable F₁ hybrids of completely male sterile plants of the corresponding Lolium species, comprising the following steps:
a) producing completely male sterile plants of the corresponding Lolium species (MSL plants) according to the method described above, and
b) back-crossing the MSL plants obtained from step a) with plants of the same Lolium species, which carry a normal fertile cytoplasm and which maintain the sterility of the MSL plants (maintainer plants).

In natural Lolium populations, a differentiated proportion of maintainer plants is present, which is determined by corresponding test crossings with investigation of the F₁ generation on sterility.

A stable MSL line is preferably obtained by repeated back-crossing with maintainer lines.

In a further preferred embodiment of the present invention, the sterility-inducing plasm of the diploid MSL line is brought to the tetraploid valence by polyploidization. The feature presentation on the tetraploid valence is analogous to the diploid valence.

The polyploidization is achieved by treatment of MSL plants with mitosis inhibitors such as for example chalones, colchicine, narcotin, quinones and the like. Especially preferred, the polyploidization is achieved by treatment with colchicine.

For example, in L. perenne and L. multiflorum there are both tetraploid and diploid forms. Because the species L. hybridum is a direct crossing of the two aforementioned species, the MSL system can also be used in both valences in this species.

Also subject of the present invention are stable MSL lines of completely male sterile plants of the genus Lolium, which have been produced by the method described above.

Furthermore, a transfer of the Lolium perenne MSL plasm to Lolium species such as Lolium hybridum and Lolium multiflorum is possible by crossings. The present invention thus provides the possibility of producing hybrid plants of the Lolium species described above on the basis of a Lolium line with stable and complete male sterility.

Both maintainer, MSL line and pollinator (=fertile father line which is used for the production of F₁ hybrids) are preferably of the same species for L. perenne and L. multiflorum. Because L. hybridum per se is a combination of the two first-mentioned species, all three components (maintainer, MSL line and pollinator) may be combined from the two basic species. Maintainers may be isolated or developed by conventional techniques from commercially available varieties and breeding strains. Any pollinator may be selected, because the fertility of the F₁ hybrids is not important, because only the vegetative mass of food grasses is used.

Using the method according to the invention MSL lines could thus be produced, which demonstrate a high degree of male sterility and stability in comparison to male sterile Lolium plants hitherto known and which thus differ substantially from these. With the help of the MSL lines according to the invention it is therefore possible for the first time to produce F₁ hybrid varieties of Lolium species, particularly the food grass species Lolium perenne, Lolium multiflorum and Lolium hybridum.

Further, the present invention provides for the first time a method by what the MSL plants may be distinguished from Lolium plants with partial or instable male sterility by Southern Blot hybridization based on the hybridization pattern.

The present invention is illustrated in the following examples, without limiting it in any way.

### EXAMPLES

### Example 1

### Mutagenesis of the Caryopses Material of Lolium Plants

The starting material for the experimental mutagenesis was a diploid L. perenne wild-type. The caryopses were incubated using the mutagenic agent N-ethyl urea (C₃H₈N₂O) in a concentration of 0.025% for 18 hours.

From the 20,000 L. perenne individual seeds treated in this way, 1200 potentially mutated individual plants (first generation after mutagenesis=M₁) could be raised. The remaining caryopses either did not germinate or the shoots were not viable.

The 1200 M₁ plants were then visually inspected for pollen distribution, whereupon 20 individual plants were at first classified as sterile. The individual plants preselected in this way were then tested by three different test methods directed to the pollen vitality (see Table 1): 1. Method according to Alexander (see above), 2. Light green reagent (\check{S}inska, see above), 3. Lugol's solution. After examination 19 individual plants were classified as partially sterile and one single plant (M 361) was classified as completely sterile.

Based on the completely sterile mutant M 361 a stable MSL line in diploid perennial ryegrass, hereinafter referred to as MSL-19, was developed by back-crossing with maintainer lines.

After polyploidization the cytoplasmic male sterility could also be established in the tetraploid valence. The tetraploid MSL line is hereinafter referred to as MSL-163.

By repeated back-crossing, the transfer of the MSL plasm from L. perenne to L. multiflorum could be achieved. Here too, MSL material was produced in diploid and tetraploid valence.

### Example 2

### Inspection of the Pollen of the Plants (Pollenbonitur)

The characterization of the male sterility of the MSL line according to the invention with complete cytoplasmic male sterility was performed by inspection of the anthers and pollen and in comparison with already known CMS (cytoplasmic male sterility) plants of Lolium perenne. As a direct comparison first the L. perenne CMS line, CMS-1, was used (D. Burkert, R. Schlenker, Pollensterilität bei Lolium perenne L. und Festuca pratensis Hunds. Wiss. Z. Univ. Rostock math.-naturwiss. R., 1975, 4.7, 845-850; I.- Gaue, Ergebnisse von Untersuchungen zur Hybridzüchtung bei Lolium perenne Tag.-Ber., Akad. Landwirtsch.-Wiss. DDR, Berlin (1981) 191, 119-126).

Surprisingly, the degree of sterility of the MSL line produced by the method according to the invention was uniformly high, irrespective of the anther shaping of the individual MSL plants, wherein additionally reliable transmission of the sterility-inducing plasm was found for MSL (see Tables 2 to 4). This distinguishes them from the sterile crossing progeny of CMS sources known from the prior art.

MSL-19 was characterised by a reduced anther size without viable pollen. The latter was also proven by the mentioned staining methods. The feature characteristic of MSL on the tetraploid valence (MSL-163) was analogous to the diploid valence.

Besides the CMS source CMS-1, the variety Inca (CMS-I1 to -I4), which is based on a CMS system, as well as plants of seven CMS sources from the Landessaatzuchtanstalt Hohenheim (CMS-112, CMS-113, CMS-114, CMS-115, CMS-117, CMS-118, CMS-119) were included in the further investigations in order to facilitate a comparison with other available Lolium CMS plants. For that, pollen of mature anthers of the individual plants CMS-1 (S), CMS-1 (N) as well as, representing MSL, the individual plants of the line MSL-163 (S) were analysed and compared with each other under two different environmental conditions, i.e. hothouse and outdoor. Moreover, the pollen of the individual plants CMS-112 to CMS-115 as well as CMS-117 to CMS-119 was inspected.

a) CMS-1 (S)

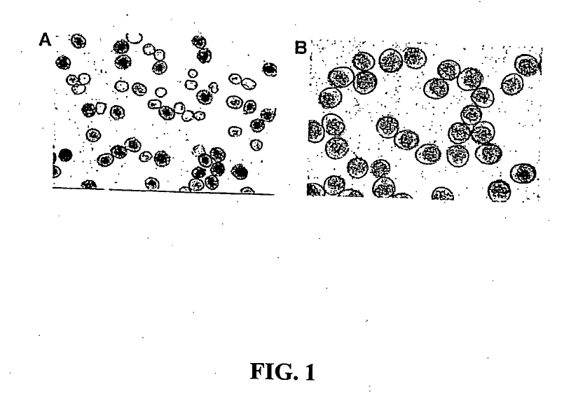

A total of 40 individual plants CMS-1 (S) were investigated. It was shown that besides completely sterile plants, also semi-sterile with percentages of fertile pollen between 20% and 75% (FIG. 1A) as well as completely fertile plants (FIG. 1B) were present.

The different ratios between fertile and sterile pollen in semi-sterile plants are most probably due to environmental factors, because individual plants produce different percentages of fertile pollen under different environmental conditions. The latter confirms the instability of the source CMS-1.

b) CMS-1 (N)

Of 34 individual plants CMS-1 (N) examined, 32 were classified as completely fertile, 2 individual plants in contrast were semi-sterile, i.e. in squeeze preparation both fertile and sterile pollen could be detected.

c) MSL-163 (S)

Within MSL-163 (S) 20 individual plants were analysed and all were classified as completely sterile. The stability of the CMS in this line could be due to an early termination of the pollen development; stainable pollen grains were not observed in any case.

d) CMS-112 to -115, CMS-17 to CMS-119

The pollen inspection of individual plants of each source showed that the pollen of the sources CMS-112, CMS-113, CMS-114, CMS-115, CMS-117, CMS-118 and CMS-119 should be classified as semi-sterile, i.e. there were stainable fertile pollen grains besides sterile pollen grains in all plants. This can be explained, as in the case of CMS-1 (S), by an instability of the cytoplasmic male sterility.

### Example 3

### Molecular Biological Analyses of MSL Plants and Comparative Plants with Cytoplasmic Male Sterility

In addition to the phenotypical data (pollen inspection of Example 2), the method of Southern hybridization was used for the reliable genotypical differentiation between MSL and known CMS systems in Lolium.

a) Isolation of Total DNA

The isolation of total DNA was performed essentially according to Wilkie (Isolation of total genomic DNA. In: M. S. Clark (Ed.) Plant Molecular Biology—A Laboratory Manual. 1997, Springer Verlag, Berlin Heidelberg New York, 3-14). In a stainless steel cylinder pre-cooled with liquid nitrogen (LN₂) 1 to 2 g of LN₂ frozen leaf material was ground with a ball mixer mill (Retsch, Haan) to a fine powder and transferred into 50 ml reaction vessels. It was then incubated in 20 ml 2×CTAB buffer in a water bath at 65° C. for 90 min, followed by two extractions with chloroform/isoamylalcohol (24:1, v/v). After centrifugation at 5000× g for 15 min in a fixed-angle rotor HFA 13.50 (Heraeus, Hanau), the aqueous phase was transferred into new 50 ml reaction vessels. The RNA was degraded by adding 1/100 vol. RNAse solution (10 mg/ml) and incubation for 30 min at 37° C. The DNA was precipitated by adding 0.7 vol.-% isopropanol and incubation at room temperature for 20 min. The DNA was transferred into 10 ml 76% ethanol/0.2 M NaOAc with the help of a Pasteur pipette and incubated for 20 min on ice. This was followed by a further washing step for 5 min in 2 ml 70% ethanol on ice. The DNA was then pelleted by centrifugation at 13000×g for 4 min (Biofuge 22 R, Heraeus). After aspiration of the excess EtOH the pellet was dried at room temperature and dissolved in TE buffer (volume depends on the pellet size). The DNA concentration was determined photometrically at 260 nm (GeneQuant I1, Pharmacia Biotech, Freiburg). All buffers used for DNA isolation are shown in Appendix A.

b) Isolation of Mitochondrial DNA

Mitochondrial DNA was isolated according to the protocols of Kiang et al. (Cytoplasmic male sterility (CMS) in Lolium perenne L.: 1. Development of a diagnostic probe for the male-sterile cytoplasm. Theor Appl Genet. 1993, 86, 781-787) and Chase and Pring (Properties of the linear N1 and N2 plasmid-like DNAs from mitochondria of cytoplasmic male-sterile Sorghum bicolor. Plant Mol Biol 1986, 6, 53-64). As starting material fresh leaf mass of hothouse plants was used that had been darkened before for 16-20 h with black foil. All working steps up to the lysis of the mitochondria were performed at 4° C. In the first working step 60 g of leaf mass were ground in 400 ml extraction buffer in a jug mixer (Gastronom GT95, W. Krannich, Göttingen), filtered through 5 layers of gauze (YPSIGAZE 8-fold, Holthaus-Medical, Remscheid) and then centrifuged for 10 min at 5000×g. The supernatant was decanted into a new centrifuge tube and centrifuged again for 10 min at 16000×g for the pelleting of the mitochondria. The mitochondrial pellet was then resuspended with the help of a sterile camel hair brush in 8 ml DNAse buffer. After adding 8 mg DNAse I, the nuclear and plastid DNA was degraded (90 min at 4° C.). In the next step the mitochondrial suspension was underlaid with 20 ml washing buffer and centrifuged at 12000×g (30 min). This was followed by a further resuspension of the pellet in washing buffer with subsequent centrifugation.

The mitochondria were lysed in 3 ml lysis buffer to which was added proteinase K (100 μg/ml final concentration) and 0.5% SDS at 37° C. (1 h). 3 ml extraction buffer was then added and the samples were incubated at 65° C. for 10 min in a water bath.

After aliquoting the samples (600 μl) in Eppendorf reaction vessels (2 ml), the proteins were precipitated by adding 200 μl 5 M potassium acetate on ice (20 min). The proteins were pelleted by centrifugation of the samples for 5 min at 13000 rpm, the supernatants were transferred into new reaction vessels and the mtDNA was precipitated by adding 400 μl isopropanol and 40 μl 5 M ammonium acetate overnight at −20° C. After centrifugation for 5 min at 13000 rpm, the mtDNA was pelleted and resuspended in 100 μl “50−10” TE buffer.

This was followed by the bringing together of several aliquots to a volume of 500 μl and the degradation of RNA by adding RNase A (100 μg/ml final concentration) for 1 h at 37° C. The mtDNA was precipitated again as described above, the DNA pellet was dissolved in 100 μl TE buffer. The concentration was determined as in the case of the total DNA. All buffers used for the isolation of mtDNA are shown in Appendix A.

c) Restriction, Electrophoresis and Blotting

For the hybridization experiments, 5 μg of total or mtDNA were restricted with 5 U restriction enzyme, adding the corresponding reaction buffers, for at least 4 h at 37° C. The endonucleases HindIII, BamHI, EcoRV, XbaI, DraI and HaeIII (Gibco BRL, Eggenstein) were used as restriction enzymes.

The DNA fragments were separated in a 1% agarose gel for 6-8 h at 50-60 V in 1×TAE buffer after adding 5×loading buffer and then stained with an ethidium bromide solution (0.1 mg/ml). The size of the fragments was determined with the aid of a DIG labelled DNA length standard (Roche, Grenzach-Wyhlen).

The DNA was then transferred to a positively loaded nylon membrane (Roche) by capillary blot. The transfer took place overnight, 20×SSC solution was used as transfer medium.

After DNA transfer the filters were washed in 2×SSC (10 min) and sealed while still moist in plastic foil until hybridization took place. For fixing the DNA a UV radiation took place (30 s; 0.120 J/cm²).

d) Description of the Probes

The primers for amplification of the gene probes required were derived from cDNA sequences of the mitochondrial genome of Arabidopsis thaliana after database searches (EMBL; ID Miatgen). Different genes which code for ribosomal proteins and subunits of the protein complexes of the respiratory chain were selected (see Table 5).

For control of contamination of isolated mtDNA with genomic DNA a ubiquitous nuclear genomic cDNA gene probe (actin) was used. The primers were derived from known actin cDNA sequences from rice; the amplification was performed with rye pollen cDNA.

e) Generation of the Probes

The primer pairs for the amplification of the probes used were derived from cDNA sequences of the corresponding genes with the help of the computer programme OLIGO 5.0 (see Table 6). The probes were labelled by incorporation of Digoxigenin-dUTP (Roche) during the PCR reaction with a thermocycler, model UNO from Biometra (Göttingen).

The reaction parameters are shown in Tables 7 and 8.

f) Non-Radioactive Southern Hybridization

For the non-radioactive Southern hybridization the DIG system from Roche was used. The reaction was performed in hybridization tubes in a hybridization oven (Stuart Scientific, Staffordshire, UK). 1-2 filters per tube were prehybridised in 20 ml DIG-Easy-Hyb (Roche) for at least 1 h. The prehybridization solution was then replaced by a new hybridization solution containing the labelled probe. Prior to adding the probe it was denatured in a water bath (100° C.) for 10 min and then incubated for 5 min on ice. 2-5 μl labelled probe DNA were used per ml hybridization solution. The hybridization was performed for at least 15 h at 39° C. The detection reaction was performed according to Roche protocols. The exposure time of the X-ray films was 10 min to 2 h, depending on the signal strength. The filters were rehybridized according to the manufacturer's instructions. The probe solutions were stored at −20° C. and used for another 8 to 10 hybridizations after denaturing at 68° C. in a water bath.

### Example 4

### Detection of mt-Specific Signals after Hybridization of Total DNA

It was first of all clarified whether using total DNA in combination with mitochondrial probes can lead to other or additional hybridization signals, which could be due to the presence of mitochondrial sequences in the nuclear genome. For this reason, mitochondrial DNA was isolated from freshly harvested leaf samples of the plants CMS-1 (N), CMS-1 (S),

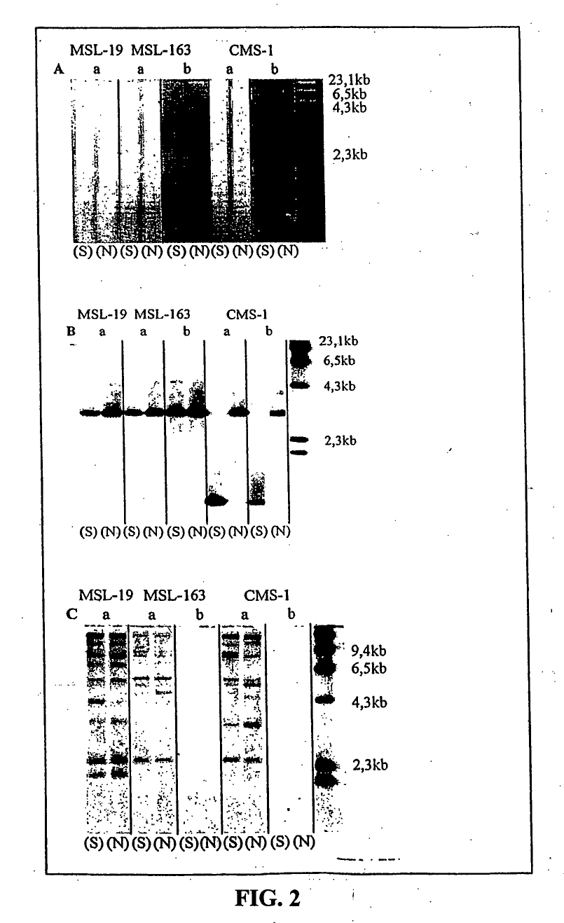

MSL-163 (N) and MSL-163 (S) to be investigated and the hybridization results were compared with those using total DNA. After restriction of total DNA and mtDNA of the same line with the restriction enzyme HindIII and electrophoretic separation of the samples in the agarose gel, no DNA could be visually detected after ethidium bromide staining. In the lanes with total DNA a continuous fragment distribution in the range of about 1 to 23 kb could be observed, which points to the total restriction of the DNA (see FIG. 2A).

After hybridization with the mt gene probe nad9 and mtDNA or total DNA as template, identical hybridization signals could be detected. The result shows that by using mt gene probes and total DNA as template, mtDNA-specific signals can be detected (see FIG. 2B).

In comparison thereto, by using a cDNA probe of the nuclear-encoded actin gene it could be shown that the mtDNA was not contaminated with genomic DNA, because as expected, only in the lanes with total DNA hybridization signals could be detected (see FIG. 2C). Because it was thus shown that the probes were mtDNA-specific, total DNA was used in futher experiments.

For more detailed molecular biological studies, the plant material was divided into groups. The first group consisted of the comparative CMS line CMS-1 (S) and the corresponding maintainer CMS-1 (N), the second group included the male sterile MSL lines MSL-163 (S) and MSL-19 (S) and the corresponding male fertile maintainer lines MSL-163 (N) and MSL-19 (N).

A total of 34 different probe/enzyme combinations were tested, 14 of which made is possible to distinguish between the male sterile lines (S) and the corresponding maintainers (N) (see Table 9).

Further, the (S) plasms of the two groups CMS and MSL could be clearly distinguished, especially by using the probe nad9 in combination with different restriction enzymes (see Table 10).

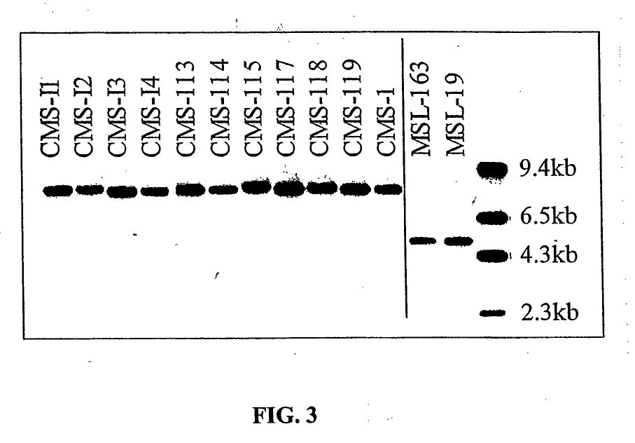

An extended set of available Lolium CMS sources was integrated into the studies with the probe/enzyme combination nad9/DraI: three plants of the CMS-Inca source (CMS-I1, CMS-I2, CMS-I3, CMS-I4), CMS-113, CMS-114, CMS-115, CMS-117, CMS-118 and CMS-119 (see FIG. 3). Here, the MSL plants MSL-163 and MSL-19 could be clearly distinguished from all other CMS sources, which for their part showed an identical hybridization pattern among themselves.

TABLE 1
Staining methods for the pollen vitality test

| Method for the differentiation | | |
|---|---|---|
| of fertile and sterile pollen | (ALEXANDER, 1969) | |
| Staining: | | |
| fertile pollen grains | dark purple | |
| sterile pollen grains | bright green | |
| Composition of the solution: | | |
| Ethanol | 10 | ml |
| Malachite green (1% in 96% alcohol) | 1 | ml |
| dist. water | 50 | ml |
| Glycerol | 25 | ml |
| Phenol | 5 | g |
| Chloral hydrate water | 5 | g |
| Fuchsin (1% in water) | 5 | ml |
| Acid Orange 10 (1% in water) | 0.5 | ml |
| Glacial acetic acid (pH 3.2) | 1-2 | ml |

| Light green | (\check{S}INSKA, 1976) |
|---|---|
| Staining: | |
| Fertile pollen grains | Dark green with clear netlike |
| | surface structure |
| Sterile pollen grains | Mostly completely colourless, |
| | and stained a weak green by |
| | degenerated plasma |
| Composition of the solution: | |
| Glycerol | 1 part |
| Lactic acid | 1 part |
| Phenol | 1 part |
| Lugol's solution | |
| Staining: | |
| Fertile pollen grains | Red staining |
| Composition of the solution: | |
| Potassium iodide | |

TABLE 2
Feature presentations of crossing progeny of the
CMS line MSL-19 and CMS-1 (both Lolium perenne)

| Feature | MSL-19 | CMS-1 |
|---|---|---|
| Valence | Diploid | Diploid |
| Generation | experimental | Spontaneous (found in |
| | mutagenesis with | assortments and propagation |
| | N-ethylurea | stocks in Gülzow in |
| | | 1969 & 1970; BURKERT |
| | | and SCHLENKER, 1975) |
| Visual sterility | very good | very good |
| Anther expression | white-green-yellow | white-green-yellow sterile |
| | sterile or mixed | or mixed colours |
| | colours | |
| Visual sterility/ | good correspondence | No correspondence |
| degree of | independently of | High degree of partially |
| sterility | anther shaping | sterile depending on the |
| | | anther shaping |
| Environmental | very low | high |
| dependence | | |

TABLE 3
Percentage of completely sterile genotypes in
F₁ crossing progeny of Lolium perenne depending
on the CMS source

| | | Number of visually | |
|---|---|---|---|
| | | sterile F₁ plants | |
| | | examined after | Relative percentage |
| CMS-Source | Mode of | backcrossing with | of completely |
| (S) | origin | maintainer (N) | sterile plants |
| CMS-1 | spontaneous | 1333 | 53.3 ^{x)} |
| CMS-INCA | Interspecific | 172 | 47.7 ^{x)} |
| | crossing | | |
| CMS-5 B | Interspecific | 225 | 77.0 ^{xx)} |
| (Irland) | crossing | | |
| MSL-19 | Mutagenesis | 1500 | 99.0 ^{xxx)} |
| | with N- | | |
| | ethylurea | | |

^{x)} Detection staining method light green reagent
^{xx)} According to Conolly (1984) sterility class 1 and 2^{v}
^{xxx)} Detection staining method Alexander
^{v} Sterility class 1: Anthers are flat, non-dehiscent, shrunken, mostly white or translucent with thin cell walls. The anthers contain no pollen. Sterility class 2: Anthers are non-dehiscent and shrunken, but not quite as flat as in class 1, anthers contain no viable pollen, some empty pollen cases are present on staining.

TABLE 4
Transmission of sterility of Lolium perenne CMS
lines MSL-19 and CMS-1; results of the sterility
test of F₁ material with identical pollinators (maintainers)

| | | |
|---|---|---|
| | | Results of the sterility |
| Crossing combination | Number of | test (relative values) |

| CMS (S) × | F₁ plants | visual | pollen |
|---|---|---|---|
| pollinator (N) | investigated | sterility | vitality ^{x} |
| MSL-19 × 85/5/9/6 | 30 | 100 | 0 |
| MSL-19 × KE 23/85 | 35 | 100 | 0 |
| CMS-1 × 85/5/9/6 | 30 | 100 | 15.20 |
| CMS-1 × KE 23/85 | 30 | 100 | 13.80 |

^{x} According to staining method of Alexander (see above)

TABLE 5
Function of the mitochondrial genes used in higher plants

| Protein | Mitochondrial gene |
|---|---|
| Subunits of cytochrome C oxidase | coxI, coxII, coxIII |
| NADH dehydrogenase | nad6 |
| NADH: Ubiquinone oxidoreductase | nad9 |
| Ribosomal proteins: large subunit | rpl6 |
| small subunit | rps3 |
| Apocytochrome b | cob |
| Cytochrome C biogenesis ORF 206 | ccb206 |

TABLE 6

Description of the primer pairs used (U = upper
primer, L = lower primer)

| | | | | |
|---|---|---|---|---|
| | | Anneal- | Am- | |
| | | ing | pli- | SEQ |
| | | tempera- | con | ID |
| Probe | Primer (5′-3′) | ture | (bp) | NO. |

| | | | | | |
|---|---|---|---|---|---|
| coxI | TTACTTCACATAGCTTTTCGTU | U | 52.1° C. | 1556 | 1 |
| | CCACAAACCACAAGGATATAG | L | | | 2 |
| coxII | CGTAAAGGCATGATTAGTTCC | U | 52.3° C. | 697 | 3 |
| | GATTGTTCTAAAATGGTTATTCCTC | L | | | 4 |
| coxIII | ATGATTGAATCTCAGAGGCAT | U | 53.0° C. | 797 | 5 |
| | CATATACCTCCCCACCAATAG | L | | | 6 |
| nad6 | TTAGTAGATCGTGAGTGGGTC | U | 51.6° C. | 563 | 7 |
| | GTGCTAAAAATCCGGTACAT | L | | | 8 |
| nad9 | TTATCCGTCGCTACGCTGTTC | U | 55.0° C. | 3392 | 9 |
| | AATGGAAAGATCGGAACATGG | L | | | 10 |
| rp15 | ATGTTTCCACTCAATTTTCAT | U | 52.2° C. | 522 | 11 |
| | GCTCCACAGTGGTAAAGTCT | L | | | 12 |
| rp16 | TTACGACCACTGAACAAACTT | U | 53.0° C. | 535 | 13 |
| | TTTAACCATAAAATCGATTATGC | L | | | 14 |
| rps3 | CTATATTTCGTACGTTTCGGA | U | 52.4° C. | 1594 | 15 |
| | TTATTATGGTAAATTTGTGTATCAA | L | | | 16 |
| cob | ATGACTATAAGGAACCAACGA | U | 52.1° C. | 1174 | 17 |
| | GATCAGTCTCATCCGTGTAA | L | | | 18 |
| ccb20 | ATGAGACGACTTTTTCTTGAA | U | 52.2° C. | 616 | 19 |
| 6 | CTTGTAAACTAATCGAGACCG | L | | | 20 |
| Actin | CACACTGTCCCCATCTATGAA | U | 57.9° C. | 650 | 21 |
| | CTCTTGGCTTAGCATTCTTGG | L | | | 22 |

TABLE 7
PCR conditions for the generation of the DNA probes used

| PCR components | Amount | |
|---|---|---|
| DIG dUTP (10×) | 5 | μl |
| d-NTP-Mix (10 mM) | 1 | μl (200 μM) |
| Primer (5 μM) | 2.5 | μl each (0.25 μM each) |
| Taq polymerase* (5 U/μl) | 0.15 | μl (0.75 U) |
| 10× reaction buffer | 5 | μl |
| MgCl₂ solution (50 mM) | 1.55 | μl (1.5 μM) |
| H₂O | 17.3 | μl |
| Template-DNA | 15 | μl (75 ng) |

Reaction volume 50 μl

*Silverstar, Eurogentec

TABLE 8
PCR conditions (30 cycles 2.-4.)

| | | | |
|---|---|---|---|
| 1. | Denaturation (single) | 94° C. | 2 min |
| 2. | Denaturation | 94° C. | 1 min |
| 3. | Annealing | see Table 6 | 1 min |
| 4. | Extension | 72° C. | 2 min |
| 5. | Extension prolongation (single) | 72° C. | 2 min |

TABLE 9
Discrimination between (S) and (N) cytoplasm of the plants MSL-19
and MSL-163 (MSL) in comparison to the plant CMS-1 (CMS)

| | Restriction enzyme | | | | | | | | | | | |
|---|---|---|---|---|---|---|---|---|---|---|---|---|
| mt | HindIII | | XbaI | | DraI | | EcoRV | | BamHI | | HaeIII | |
| gene | MSL | | MSL | | MSL | | MSL | | MSL | | MSL | |
| probe | CMS | | CMS | | CMS | | CMS | | CMS | | CMS | |
| coxI | + | + | n.d. | | + | + | n.d. | | + | + | n.d. | |
| coxIII | + | + | n.d. | | + | + | + | + | + | + | n.d. | |
| nad6 | + | + | n.d. | | + | + | − | − | + | + | − | − |
| nad9 | − | + | − | + | + | + | − | + | + | + | − | + |
| ccb206 | − | − | − | − | − | − | + | − | − | − | − | − |
| rpl5 | − | − | n.d. | | n.d. | | − | − | n.d. | | n.d. | |
| rpl6 | + | − | − | − | + | + | n.d. | | + | − | n.d. | |
| cob | n.d. | | + | − | n.d. | | + | − | n.d. | | + | − |
| rps3 | n.d. | | n.d. | | n.d. | | + | + | n.d. | | n.d. | |

MSL = DNA bulk of the plants MSL-19 and MSL-163
CMS = male sterile plant CMS-1
+ = Discrimination between (N)- and (S) cytoplasm is possible
− = Discrimination between (N)- and (S) cytoplasm is not possible
n.d. = not determined probe/enzyme combinations

TABLE 10
Hybridization pattern of the (S) cytoplasms of the plants MSL-19
and MSL-163 (MSL) in comparison to the plant CMS-1 (CMS)

| | (b) Restriction enzyme | | | | | | | | | | | |
|---|---|---|---|---|---|---|---|---|---|---|---|---|
| | HindIII | | XbaI | | DraI | | EcoRV | | BamHI | | HaeIII | |
| mt gene | MSL | | MSL | | MSL | | MSL | | MSL | | MSL | |
| probe | CMS | | CMS | | CMS | | CMS | | CMS | | CMS | |
| CoxI | 1 | 2 | n.d. | | 1 | 2 | n.d. | | 1 | 1 | n.d. | |
| coxIII | 1 | 2 | n.d. | | 1 | 2 | 1 | 2 | 1 | 1 | n.d. | |
| nad6 | 1 | 2 | n.d. | | 1 | 1 | 1 | 1 | 1 | 2 | 1 | 1 |
| nad9 | 1 | 2 | 1 | 2 | 1 | 2 | 1 | 2 | 1 | 2 | 1 | 2 |
| ccb206 | 1 | 1 | 1 | 1 | 1 | 1 | 1 | 2 | 1 | 1 | 1 | 1 |
| rpl5 | 1 | 1 | n.d. | | n.d. | | 1 | 1 | n.d. | | n.d. | |
| rpl6 | 1 | 1 | 1 | 1 | 1 | 1 | n.d. | | 1 | 1 | n.d. | |
| cob | n.d. | | 1 | 2 | n.d. | | 1 | 1 | n.d. | | 1 | 2 |
| rps3 | n.d. | | n.d. | | n.d. | | 1 | 1 | n.d. | | n.d. | |

MSL = Plants MSL-19 and MSL-163
CMS = Male sterile plant CMS-1
n.d. = not determined
Matching numbers within a probe/enzyme combination indicate identical hybridization patterns.

APPENDIX A

| Isolation of total DNA: | |
|---|---|
| 2 × CTAB | TE buffer |
| 0.1 M Tris/HCl pH 8.0 | 10 mM Tris/HCl pH 8.0 |
| 1.4 M NaCl | 1 mM EDTA pH 8.0 |
| 0.5 M EDTA pH 8.0 | |
| 2% CTAB | |
| 0.04 M 2-mercaptoethanol | |
| Isolation of mtDNA | |
| Extraction buffer 1 | DNase buffer |
| 0.44 M Sucrose | 0.44 M Sucrose |
| 50 mM Tris pH 8.0 | 50 mM Tris pH 8.0 |
| 3 mM EDTA pH 8.0 | 10 mM MgCl₂ |
| 0.5% BSA | |
| 10 mM 2-mercaptoethanol | |
| Washing buffer | Lysis buffer |
| 0.6 M Sucrose | 50 mM Tris |
| 25 mM EDTA pH 8.0 | 20 mM EDTA |
| 50 mM Tris pH 8.0 | 0.5% SDS |
| | pH 8.0 |
| Extraction buffer 2 | “50-10” TE buffer |
| 0.15 M Tris | 50 mM Tris |
| 0.1 M NaCl | 10 mM EDTA |
| 80 mM EDTA | pH 8.0 |
| 1.5% SDS | |
| pH 8.0 | |
| TE buffer | |
| 10 mM TRIS | |
| 1 mM EDTA | |
| pH 8.0 | |
| Electrophoresis and blotting | |
| 1× TAE | 20× SSC |
| 40 mM Tris/acetate | 3 M NaCl |
| 1 mM EDTA | 0.3 M Na-citrate |
| | pH 7.0 |

5× loading buffer
12.5% Ficoll
62.5 mM EDTA pH 8.0
0.5% SDS
5× TAE
0.02% bromphenol blue
0.02% xylene cyanol

What is claimed is:

1. A method for the production of stable F, hybrids of completely male sterile plants of the genus Lolium, comprising the following steps:
a) producing completely male sterile plants of the genus Lolium (MSL plants) by the steps of:
i) mutagenizing caryopses material of wild-type plants of the genus Lolium;
ii) examining the mutagenized Lolium plants by Southern Blot techniques; and
iii) identifying completely male sterile Lolium plants, and
b) back-crossing the MSL plants obtained in step a) with plants of the genus Lolium, which have normal fertile cytoplasm and which maintain the sterility of the MSL plants (maintainer plants).

2. The method according to claim 1, wherein the mutagenesis is performed by addition of a chemical mutagen.

3. The method according to claim 2, wherein the chemical mutagen is N-ethyl urea.

4. The method according to claim 1, wherein the Lolium plants are selected from the group consisting of Lolium perenne, Lolium multiflorum and Lolium hybridum.

5. The method according to claim 1, wherein the Southern Blot techniques employ primer pairs for the amplification of the probes used for the Southern Blot hybridization, wherein the primer pairs are selected from the group consisting of the following primer pairs:

| | | |
|---|---|---|
| a) | TTACTTCACATAGCTTTTCGTU | (SEQ ID No. 1) |
| | CCACAAACCACAAGGATATAG | (SEQ ID No. 2) |
| b) | ATGATTGAATCTCAGAGGCAT | (SEQ ID No. 5) |
| | CATATACCTCCCCACCAATAG | (SEQ ID No. 6) |
| c) | TTAGTAGATCGTGAGTGGGTC | (SEQ ID No. 7) |
| | GTGCTAAAAATCCGGTACAT | (SEQ ID No. 8) |
| d) | TTATCCGTCGCTACGCTGTTC | (SEQ ID No. 9) |
| | AATGGAAAGATCGGAACATGG | (SEQ ID No. 10) |
| e) | ATGACTATAAGGAACCAACGA | (SEQ ID No. 17) |
| | GATCAGTCTCATCCGTGTAA | (SEQ ID No. 18) |
| f) | ATGAGACGACTTTTTCTTGAA | (SEQ ID No. 19) |
| | CTTGTAAACTAATCGAGACCG, | (SEQ ID No. 20) |

further wherein the sequences are shown in 5′-3′ direction and further wherein the first sequence is the upper primer and further wherein the probes generated by amplification with the primers are used for Southern Blot analysis in one of the following combinations with restriction enzymes:
a) together with HindIII or DraI
b) together with HindIlI, DraI or EcoRV
c) together with HindIII or BamHI
d) together with HindIII, XbaI, Dral, EcoRV, BamHI or HaeIII
e) together with XbaI or HaeIII
f) together with EcoRV.

6. The method according to claim 1,
wherein plants of the corresponding species are used as maintainer plants, which lead to a 100% pollen-sterile progeny after crossing with the MSL line.

7. The method according to claim 1,
wherein a multiple back-crossing with maintainer plants is performed.

8. The method according to claim 1,
wherein the sterility-inducing plasm of the MSL plant produced in step a) is brought to a preferably tetraploid valence by polyploidisation.

9. The method according to claim 8,
wherein the polyploidisation is achieved by treatment with colchicine.

10. Plants of the genus Lolium with complete male sterility, produced according to the method of claim 1.

11. A method for the production of hybrids with pollinator plants having normal male fertility, using the completely male sterile plants of the genus Lolium according to claim 10.

12. A hybrid seed produced by the method of claim 1.

13. Plants of the genus Lolium with complete male sterility which can be distinguished from plants with instable or incomplete male sterility by Southern Blot techniques, wherein the Southern Blot techniques employ primer pairs for the amplification of the probes used for the Southern Blot hybridisation, wherein the primer pairs are selected from the group consisting of the following primer pairs:

| | | |
|---|---|---|
| a) | TTACTTCACATAGCTTTTCGTU | (SEQ ID No. 1) |
| | CCACAAACCACAAGGATATAG | (SEQ ID No. 2) |
| b) | ATGATTGAATCTCAGAGGCAT | (SEQ ID No. 5) |
| | CATATACCTCCCCACCAATAG | (SEQ ID No. 6) |
| c) | TTAGTAGATCGTGAGTGGGTC | (SEQ ID No. 7) |
| | GTGCTAAAAATCCGGTACAT | (SEQ ID No. 8) |
| d) | TTATCCGTCGCTACGCTGTTC | (SEQ ID No. 9) |
| | AATGGAAAGATCGGAACATGG | (SEQ ID No. 10) |
| e) | ATGACTATAAGGAACCAACGA | (SEQ ID No. 17) |
| | GATCAGTCTCATCCGTGTAA | (SEQ ID No. 18) |
| f) | ATGAGACGACTTTTTCTTGAA | (SEQ ID No. 19) |
| | CTTGTAAACTAATCGAGACCG, | (SEQ ID No. 20) |

further wherein the sequences are shown in 5′-3′ direction and further wherein the first sequence is the upper primer and further wherein the probes generated by amplification with the primers are used for Southern Blot analysis in one of the following combinations with restriction enzymes:
a) together with HindIII or DraI
b) together with HindIII, DraI or EcoRV
c) together with HindIII or BamHI
d) together with HindIII, XbaI, DraI, EcoRV, BamHI or HaeIII
e) together with XbaI or HaeIIl
f) together with EcoRV.

14. A method for distinguishing MSL plants from Lolium plants with partial or instable male sterility by Southern Blot hybridization, wherein the Southern Blot hybridization employs primer pairs for the amplification of the probes used for the Southern Blot hybridisation, wherein the primer pairs are selected from the group consisting of the following primer pairs:

| | | |
|---|---|---|
| a) | TTACTTCACATAGCTTTTCGTU | (SEQ ID No. 1) |
| | CCACAAACCACAAGGATATAG | (SEQ ID No. 2) |
| b) | ATGATTGAATCTCAGAGGCAT | (SEQ ID No. 5) |
| | CATATACCTCCCCACCAATAG | (SEQ ID No. 6) |
| c) | TTAGTAGATCGTGAGTGGGTC | (SEQ ID No. 7) |
| | GTGCTAAAAATCCGGTACAT | (SEQ ID No. 8) |
| d) | TTATCCGTCGCTACGCTGTTC | (SEQ ID No. 9) |
| | AATGGAAAGATCGGAACATGG | (SEQ ID No. 10) |
| e) | ATGACTATAAGGAACCAACGA | (SEQ ID No. 17) |
| | GATCAGTCTCATCCGTGTAA | (SEQ ID No. 18) |
| f) | ATGAGACGACTTTTTCTTGAA | (SEQ ID No. 19) |
| | CTTGTAAACTAATCGAGACCG, | (SEQ ID No. 20) |

further wherein the sequences are shown in 5′-3′ direction and further wherein the first sequence is the upper primer and further wherein the probes generated by amplification with the primers are used for Southern Blot analysis in one of the following combinations with restriction enzymes:
a) together with HindIII or DraI
b) together with HindIII, DraI or EcoRV
c) together with HindIII or BamHI
d) together with HindIII, XbaI, DraI, EcoRV, BamHI or HaeIII
e) together with XbaI or HaeIII
f) together with EcoRV.


SEQUENCE LISTING (22 sequences)

SEQ ID NO: 1 - DNA, 22 bp, Artificial Sequence
  Oligonucleotide primer
ttacttcacatagcttttcgtu

SEQ ID NO: 2 - DNA, 21 bp, Artificial Sequence
  Oligonucleotide primer
ccacaaaccacaaggatatag

SEQ ID NO: 3 - DNA, 21 bp, Artificial Sequence
  Oligonucleotide primer
cgtaaaggcatgattagttcc

SEQ ID NO: 4 - DNA, 25 bp, Artificial Sequence
  Oligonucleotide primer
gattgttctaaaatggttattcctc

SEQ ID NO: 5 - DNA, 21 bp, Artificial Sequence
  Oligonucleotide primer
atgattgaatctcagaggcat

SEQ ID NO: 6 - DNA, 21 bp, Artificial Sequence
  Oligonucleotide primer
catatacctccccaccaatag

SEQ ID NO: 7 - DNA, 21 bp, Artificial Sequence
  Oligonucleotide primer
ttagtagatcgtgagtgggtc

SEQ ID NO: 8 - DNA, 20 bp, Artificial Sequence
  Oligonucleotide primer
gtgctaaaaatccggtacat

SEQ ID NO: 9 - DNA, 21 bp, Artificial Sequence
  Oligonucleotide primer
ttatccgtcgctacgctgttc

SEQ ID NO: 10 - DNA, 21 bp, Artificial Sequence
  Oligonucleotide primer
aatggaaagatcggaacatgg

SEQ ID NO: 11 - DNA, 21 bp, Artificial Sequence
  Oligonucleotide primer
atgtttccactcaattttcat

SEQ ID NO: 12 - DNA, 20 bp, Artificial Sequence
  Oligonucleotide primer
gctccacagtggtaaagtct

SEQ ID NO: 13 - DNA, 21 bp, Artificial Sequence
  Oligonucleotide primer
ttacgaccactgaacaaactt

SEQ ID NO: 14 - DNA, 23 bp, Artificial Sequence
  Oligonucleotide primer
tttaaccataaaatcgattatgc

SEQ ID NO: 15 - DNA, 21 bp, Artificial Sequence
  Oligonucleotide primer
ctatatttcgtacgtttcgga

SEQ ID NO: 16 - DNA, 25 bp, Artificial Sequence
  Oligonucleotide primer
ttattatggtaaatttgtgtatcaa

SEQ ID NO: 17 - DNA, 21 bp, Artificial Sequence
  Oligonucleotide primer
atgactataaggaaccaacga

SEQ ID NO: 18 - DNA, 20 bp, Artificial Sequence
  Oligonucleotide primer
gatcagtctcatccgtgtaa

SEQ ID NO: 19 - DNA, 21 bp, Artificial Sequence
  Oligonucleotide primer
atgagacgactttttcttgaa

SEQ ID NO: 20 - DNA, 21 bp, Artificial Sequence
  Oligonucleotide primer
cttgtaaactaatcgagaccg

SEQ ID NO: 21 - DNA, 21 bp, Artificial Sequence
  Oligonucleotide primer
cacactgtccccatctatgaa

SEQ ID NO: 22 - DNA, 21 bp, Artificial Sequence
  Oligonucleotide primer
ctcttggcttagcattcttgg